(19) United States
(12) Patent Application Publication
(10) Pub. No.: US 2024/0197340 A1
(43) Pub. Date: Jun. 20, 2024

(54) ARTHROPLASTY DEVICES, SYSTEMS, INSTRUMENTS, AND METHODS

(71) Applicant: CATALYST ORTHOSCIENCE INC., Naples, FL (US)

(72) Inventors: Steven Scott GOLDBERG, Naples, FL (US); Zachary R. LEITZE, St. George, UT (US); Ephraim AKYUZ, Salt Lake City, UT (US); Logan MORTON, Gainesville, FL (US); Dale DAVISON, Naples, FL (US)

(73) Assignee: CATALYST ORTHOSCIENCE INC., Naples, FL (US)

(21) Appl. No.: 18/287,263

(22) PCT Filed: Apr. 18, 2022
(86) PCT No.: PCT/US2022/025240

§ 371 Date: Oct. 17, 2023

Related U.S. Application Data
(60) Provisional application No. 63/176,276, filed on Apr. 17, 2021.

(51) Int. Cl. A61B 17/17; A61B 17/16; A61B 17/56; A61B 90/00; A61F 2/40; A61F 2/46

(52) U.S. Cl. CPC A61B 17/1778; A61B 17/1684; A61F 2/4003; A61F 2/4612; A61F 2/4684; A61B 2017/564; A61B 2090/034

(57) ABSTRACT

A bone reaming system may include a reamer guide plate and a reamer head. The reamer guide plate may include a guide plate body having a bone-facing surface, a superior surface opposite the bone-facing surface, and a reamer guide bore formed through the guide plate body. The reamer head may include a distal cutting surface with one or more cutting features, as well as one or more stop members coupled to a proximal end of the reamer head. The reamer guide bore may be shaped to receive and guide the distal cutting surface of the reamer head placed through the reamer guide bore. The one or more stop members may be configured to contact the superior surface of the guide plate body to limit a predetermined depth of the distal cutting surface projecting from the bone-facing surface of the guide plate body.

### TECHNICAL FIELD

The present disclosure relates to joint arthroplasty devices, systems, instruments, and methods. More specifically, the present disclosure relates to improved devices, systems, instruments, and methods for performing joint arthroplasty procedures. While portions of the present disclosure are made in the context of humeral and glenoid devices, systems, instruments, and methods for shoulder arthroplasty, the disclosed principles are applicable to arthroplasty devices, systems, instruments, and methods for other locations as well.

### BACKGROUND

Joint arthroplasty procedures are conducted to restore the function of an unhealthy joint. Typically, these procedures involve replacing the unhealthy natural articulating surfaces of the joint with artificial articulating surfaces. The new artificial articulating surfaces are typically anchored into the adjacent bones to maintain long term stability.

In shoulder arthroplasty, a humeral implant is attached to the humerus, and a glenoid implant is attached to the glenoid or scapula. There are two different main categories of shoulder arthroplasty: anatomic and reverse. In an anatomic procedure, the implant designs are intended to replicate the natural anatomy. The humeral head is replaced with a similarly shaped convex hemispherical surface, while the glenoid is replaced with a shallow concave socket. In a reverse procedure, the natural ball and socket is reversed. The humeral head is replaced with a socket fixed to the humerus and the glenoid is replaced with a ball (or glenosphere) fixed to the scapula.

Regardless of the type of procedure, fixation of the humeral component into the humerus typically involves an implant with a shaft portion that extends into the metaphysis and optionally into the diaphysis of the humerus. The goals of these implants are to preserve as much native bone as possible, maximize the mechanical stability of the implants, and allow for more physiological loading of the bone to preserve long-term fixation.

Portions of the present disclosure are made in the context of humeral and glenoid devices, systems, instruments, and methods for shoulder arthroplasty. Other applications may include femoral devices, systems, instruments, and methods for hip or knee arthroplasty, tibial devices, systems, instruments, and methods for knee or ankle arthroplasty, or other devices, systems, instruments, and methods for the elbow, wrist, hand, foot, etc.

### SUMMARY

The various arthroplasty devices, systems, instruments, and methods of the present disclosure have been developed in response to the present state of the art, and in particular, in response to the problems and needs in the art that have not yet been fully solved by currently available arthroplasty devices, systems, instruments, and methods.

In some embodiments, a reamer guide plate may include a guide plate body, a handle, and one or more fixation elements. The guide plate body may include a bone-facing surface, a superior surface opposite the bone-facing surface, and a reamer guide bore formed through the guide plate body. The reamer guide bore may be oriented to pass through the superior surface and the bone-facing surface of the guide plate body. The reamer guide bore may be shaped to receive and guide a reamer head that is placed therethrough. The one or more fixation elements may be configured to couple the bone-facing surface of the reamer guide plate to a bone.

In some embodiments, the reamer guide bore may include a circular shape defined by an annular inner side wall intermediate the bone-facing surface and the superior surface.

In some embodiments, the reamer guide plate may include one or more beveled surfaces intermediate the annular inner side wall and the superior surface.

In some embodiments, the reamer guide plate may include one or more windows formed through the guide plate body about the reamer guide bore.

In some embodiments, the one or more windows may include one or more lobe shapes adjacent the reamer guide bore.

In some embodiments, the one or more fixation elements may include one or more spikes projecting from the bone-facing surface.

In some embodiments, the handle may be removably couplable with the guide plate body.

In some embodiments, the handle may be integrally formed with the guide plate body.

In some embodiments, the reamer guide plate may include at least one guard member coupled with the reamer guide plate.

In some embodiments, a bone reaming system may include a reamer guide plate and a reamer head. The reamer guide plate may include a guide plate body having a bone-facing surface, a superior surface opposite the bone-facing surface, and a reamer guide bore formed through the guide plate body. The reamer head may include a distal cutting surface having one or more cutting features, as well as one or more stop members coupled to a proximal end of the reamer head. The reamer guide bore may be shaped to receive and guide the distal cutting surface of the reamer head placed through the reamer guide bore. The one or more stop members may be configured to contact the superior surface of the guide plate body to limit a predetermined depth of the distal cutting surface projecting from the bone-facing surface of the guide plate body.

In some embodiments, the one or more stop members may include a plurality of tabs projecting from the proximal end of the reamer head.

In some embodiments, the one or more stop members may include a first beveled surface circumscribing the proximal end of the reamer head. The reamer guide plate may also include at least one second beveled surface shaped to contact the first beveled surface of the reamer head and limit the predetermined depth of the distal cutting surface projecting from the bone-facing surface of the guide plate body.

In some embodiments, the reamer guide bore formed through the guide plate body may include a circular shape defined by an annular inner side wall intermediate the bone-facing surface and the superior surface.

In some embodiments, the reamer guide plate may include one or more windows formed through the guide plate body about the reamer guide bore.

In some embodiments, a method of reaming a bone may include: placing a bone-facing surface of a reamer guide plate against the bone; inserting a distal cutting surface of a reamer head through a reamer guide bore that is formed through the reamer guide plate, the reamer guide bore shaped to receive and guide the distal cutting surface placed therethrough; and reaming the bone with the distal cutting surface of the reamer head to form a bone cavity having a predetermined depth relative to the bone-facing surface of the reamer guide plate.

In some embodiments, the method may also include coupling the bone-facing surface of the reamer guide plate to the bone by coupling one or more fixation elements of the reamer guide plate to the bone.

In some embodiments of the method, coupling the bone-facing surface of the reamer guide plate to the bone may include pressing one or more spikes that project from the bone-facing surface into the bone.

In some embodiments, the method may also include limiting the predetermined depth of the bone cavity relative to the bone-facing surface of the reamer guide plate by contacting one or more stop members coupled to a proximal end of the reamer head with a superior surface of the reamer guide plate to limit the predetermined depth of the distal cutting surface projecting from the bone-facing surface of the reamer guide plate.

In some embodiments of the method, the one or more stop members may include a plurality of tabs projecting from the proximal end of the reamer head.

In some embodiments of the method, the one or more stop members may include a first beveled surface circumscribing the proximal end of the reamer head. The reamer guide plate may also include at least one second beveled surface shaped to contact the first beveled surface of the reamer head and limit the predetermined depth of the distal cutting surface projecting from the bone-facing surface of the guide plate body.

These and other features and advantages of the present disclosure will become more fully apparent from the following description and appended claims or may be learned by the practice of the devices, systems, instruments, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings in which:

FIG. 1A illustrates an oblique view of a humeral stem, according to an embodiment of the present disclosure; FIG. 1B illustrates another oblique view of the humeral stem of FIG. 1A; FIG. 1C illustrates a view perpendicular to a bottom surface of a socket of the humeral stem of FIG. 1A; FIG. 1D illustrates a medial view of the humeral stem of FIG. 1A; FIG. 1E illustrates an anterior view of the humeral stem of FIG. 1A; FIG. 1F illustrates a lateral view of the humeral stem of FIG. 1A;

FIG. 2A illustrates an oblique view of a convex articular component, according to an embodiment of the present disclosure; FIG. 2B illustrates another oblique view of the convex articular component of FIG. 2A; FIG. 2C illustrates a side view of the convex articular component of FIG. 2A;

FIG. 2D illustrates a cross-sectional view of the convex articular component shown in FIG. 2C;

FIG. 3A illustrates an oblique view of a baseplate, according to an embodiment of the present disclosure; FIG. 3B illustrates another oblique view of the baseplate of FIG. 3A; FIG. 3C illustrates a side view of the baseplate of FIG. 3A; FIG. 3D illustrates a cross-sectional view of the baseplate shown in FIG. 3C;

FIG. 4A illustrates an oblique view of an augmented baseplate, according to another embodiment of the present disclosure; FIG. 4B illustrates another oblique view of the augmented baseplate of FIG. 4A; FIG. 4C illustrates a side view of the augmented baseplate of FIG. 4A; FIG. 4D illustrates a cross-sectional view of the augmented baseplate shown in FIG. 4C;

FIG. 5 illustrates an exploded view of a baseplate assembly, according to an embodiment of the present disclosure;

FIG. 6 illustrates an exploded view of a baseplate assembly, according to another embodiment of the present disclosure;

FIG. 7 illustrates an oblique view of the baseplate assembly of FIG. 5, after assembly;

FIG. 8 illustrates an oblique view of the baseplate assembly of FIG. 6, after assembly;

FIG. 9A illustrates an oblique view of a reamer guide plate, according to an embodiment of the present disclosure; FIG. 9B illustrates another oblique view of the reamer guide plate of FIG. 9A;

FIG. 9C illustrates a side view of the reamer guide plate of FIG. 9A; FIG. 9D illustrates a cross-sectional view of the reamer guide plate shown in FIG. 9C;

FIG. 10A illustrates an oblique view of a reamer guide plate, according to another embodiment of the present disclosure; FIG. 10B illustrates another oblique view of the reamer guide plate of FIG. 10A; FIG. 10C illustrates a side view of the reamer guide plate of FIG. 10A; FIG. 10D illustrates a cross-sectional view of the reamer guide plate shown in FIG. 10C;

FIG. 11A illustrates an oblique view of a reamer head, according to an embodiment of the present disclosure; FIG. 11B illustrates another oblique view of the reamer head of FIG. 11A; FIG. 11C illustrates a distal end view of the reamer head of FIG. 11A; FIG. 11D illustrates a proximal end view of the reamer head of FIG. 11A;

FIG. 12A illustrates an oblique view of the reamer guide plate of FIG. 9A coupled to a humerus; FIG. 12B illustrates an oblique view of the reamer head of FIG. 11A and the humerus of FIG. 12A prior to a reaming procedure; FIG. 12C illustrates an oblique view of the reamer head of FIG. 11A inserted into the humerus of FIG. 12A during the reaming procedure; FIG. 12D illustrates an oblique view of the humerus of FIG. 12A after the reaming procedure has been performed;

FIG. 13A illustrates an oblique view of a drill guide, according to an embodiment of the present disclosure; FIG. 13B illustrates another oblique view of the drill guide of FIG. 13A; FIG. 13C illustrates a side view of the drill guide of FIG. 13A; FIG. 13D illustrates a cross-sectional view of the drill guide shown in FIG. 13C;

FIG. 14A illustrates an oblique view of a drill tool including a torque connection interface; FIG. 14B illustrates an oblique view of the drill tool of FIG. 14A including a T-handle;

FIG. 15A illustrates an oblique view of the drill guide of FIG. 13A above a humerus prior to a drilling procedure; FIG. 15B illustrates an oblique view of the drill guide of FIG. 13A coupled to the humerus of FIG. 15A; FIG. 15C illustrates an oblique view of the drill tool of FIG. 14A being inserted through the drill guide coupled to the humerus shown in FIG. 15B; FIG. 15D illustrates an oblique view of the drill tool of FIG. 14A fully inserted into the humerus of FIG. 15C to perform a drilling procedure;

FIG. 16A illustrates an oblique view of a trial humeral stem, according to an embodiment of the present disclosure; FIG. 16B illustrates another oblique view of the trial humeral stem of FIG. 16A;

FIG. 17A illustrates an oblique view of a trial concave articular component, according to an embodiment of the present disclosure; FIG. 17B illustrates another oblique view of the trial concave articular component of FIG. 17A; FIG. 17C illustrates a side view of the trial concave articular component of FIG. 17A; FIG. 17D illustrates a cross-sectional view of the trial concave articular component shown in FIG. 17C;

FIG. 18 illustrates a side view of the trial concave articular component of FIG. 17A above the trial stem of FIG. 16A, prior to assembly; FIG. 18B illustrates an oblique view of the trial concave articular component and the trial stem shown in FIG. 18A, after assembly;

FIG. 19A illustrates an oblique view of another drill guide, according to an embodiment of the present disclosure; FIG. 19B illustrates another oblique view of the drill guide of FIG. 19A;

FIG. 20A illustrates an oblique view of a reamer tool, according to an embodiment of the present disclosure; FIG. 20B illustrates another oblique view of the reamer tool of FIG. 20A;

FIG. 21A illustrates a side view of an offset reamer tool, according to another embodiment of the present disclosure; FIG. 21B illustrates an oblique view of the distal end of the offset reamer tool of FIG. 21A;

FIG. 22A illustrates an oblique view of the drill guide of FIG. 19A coupled to a glenoid cavity of a scapula; FIG. 22B illustrates an oblique view of the drill guide and scapula of FIG. 22A with a drill bit inserted through a first passageway of the drill guide and into the scapula along a first trajectory; FIG. 22C illustrates an oblique view of the scapula of FIG. 22B with the drill guide removed and the reamer of FIG. 20A inserted over the drill bit to ream the glenoid cavity along the first trajectory; FIG. 22D illustrates an oblique view of the scapula and prepared glenoid cavity from FIG. 22C being coupled to the prepared glenoid cavity with the bone screw of FIG. 5 and a driver tool.

FIG. 23A illustrates an oblique view of the drill guide of FIG. 19A coupled to a glenoid cavity of a scapula; FIG. 23B illustrates an oblique view of the drill guide and scapula of FIG. 23A with a drill bit inserted through a second passageway of the drill guide and into the scapula along a second trajectory; FIG. 23C illustrates an oblique view of the scapula of FIG. 23B with the drill guide removed and the reamer of FIG. 21A inserted over the drill bit to ream the glenoid cavity along the second trajectory; and FIG. 23D illustrates an oblique view of the scapula and prepared glenoid cavity from FIG. 23C with the augmented baseplate of FIG. 4A being coupled to the prepared glenoid cavity with the bone screw of FIG. 5 and a driver tool.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the implants, devices, systems, instruments, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

In this specification, standard shoulder anatomical terms are employed with their ordinary and customary meanings.

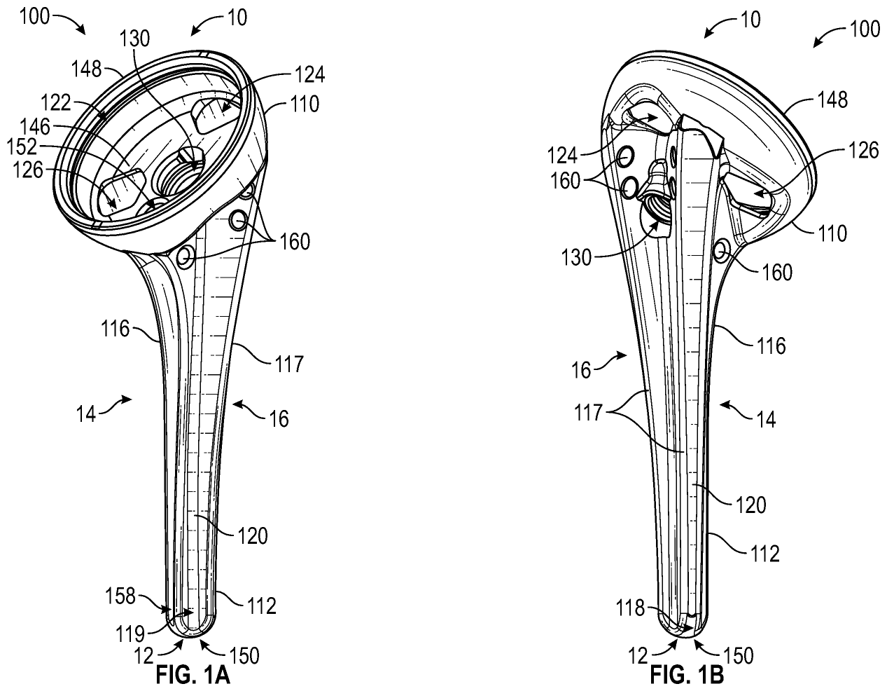

Referring to FIGS. 1A-1F, a humeral implant or humeral stem 100 may include a proximal body 110 and a distal shaft 112. In an embodiment, the distal shaft 112 may be integrally formed with the proximal body 110 to form a one-piece humeral stem. However, in other embodiments the distal shaft 112 and proximal body 110 may be removably couplable with each other.

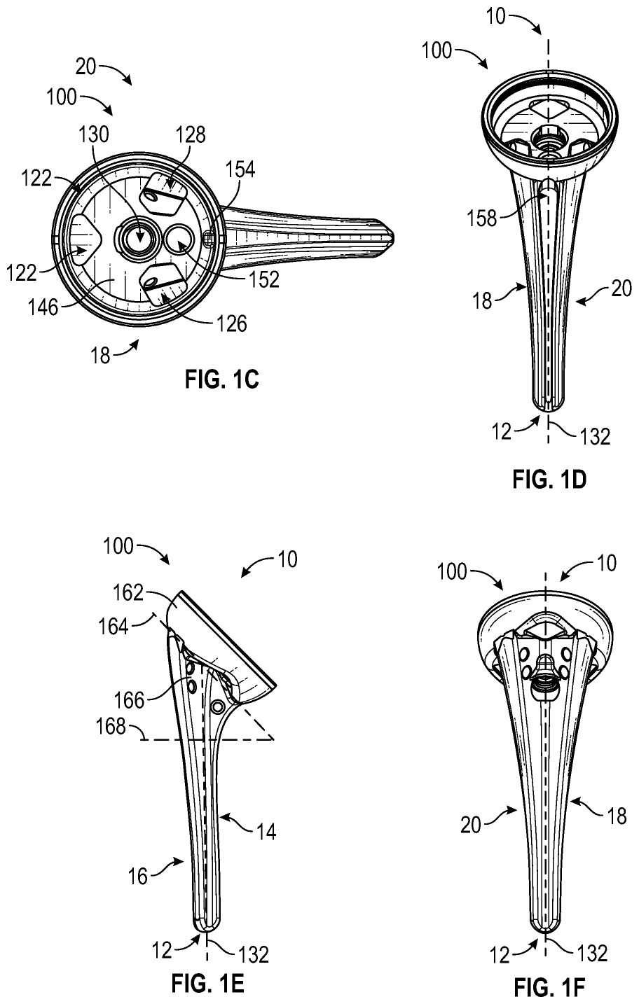

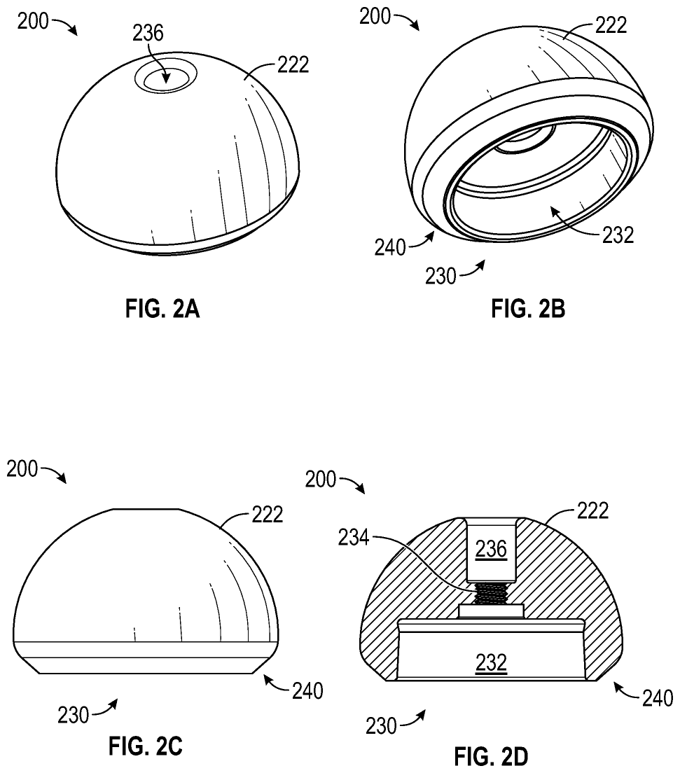

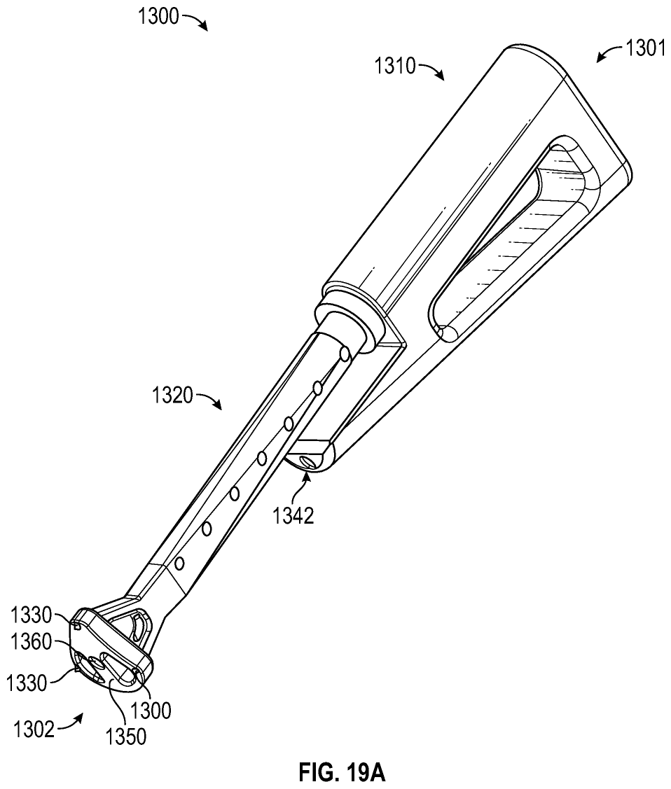

The proximal body 110 may have a convex hemispherical exterior shape and an interior socket 122 with features shaped to receive an articular component, such as a concave articular component (e.g., similar in shape to that shown in FIGS. 17A-17D) or a convex articular component (e.g., see FIGS. 2A-2D). The interior socket 122 may include a flat bottom surface 146 which may be parallel to a proximal rim 148 of the proximal body 110. The interior socket 122 may be referred to as an articular component interconnection or interface. One or more holes may extend through the proximal body 110 (e.g., see holes 124, 126, 128, 130, etc.). Holes 124, 126, 128 are shown with non-circular cross-sectional shapes which may be based upon the geometry of the corresponding ridges 116, 117 and grooves 118, 119 in the vicinity of the holes. Hole 130 is shown with a circular cross-sectional shape (see FIG. 1C) and may be internally threaded. The holes 124, 126, 128, and/or 130 may provide means or openings for inserting instruments to aid in removing or extracting the implant from the bone after implantation. Holes 124, 126, 128 may enhance rotational stability of the humeral stem 100, initially due to their distal edges digging into adjacent bone, and long-term due to bone growth proximally into the holes to the extent permitted by the articular component. Additionally, optionally, hole 130 may be included in an inserter interconnection or interface of the interior socket 122 for connection to an inserter instrument (not shown) for inserting and/or impacting the humeral stem 100 into a bone, such as a proximal humerus. A medial blind hole 152 may extend into the bottom surface 146 and partway through the proximal body 110. A medial tab 154 may project from the bottom surface 146 and/or from an inner wall of the interior socket 122. The medial tab 154 may engage with a corresponding groove in an articular component placed within the interior socket 122 to enhance rotational stability.

The distal shaft 112 may extend distally from the exterior of the proximal body 110 to terminate at a free end 150 at the distal end 12. The distal shaft 112 may start out the same size or similar in size to the proximal body and may become smaller farther from the proximal body, towards the distal end 12. In other words, the distal shaft 112 may have a larger overall outer diameter at or near proximal body 110 and a smaller overall outer diameter farther from the proximal body, near the free end 150. The distal shaft 112 may include alternating longitudinal ridges and longitudinal grooves. The ridges may be described as arms, bars, beams, branches, columns, cylinders, fins, legs, limbs, lobes, pillars, rails, ribs, shafts, struts, or other geometrical shapes. This arrangement may enhance rotational stability along most or all of the length of the distal shaft 112 when the humeral stem 100 is implanted in a proximal humerus. The distal shaft 112 of humeral stem 100 has three ridges and three grooves, although any number of ridges and grooves may be present. The illustrated arrangement of three ridges and three grooves gives the distal shaft 112 a cross-sectional shape that may be described as tri-lobed, triangular, or Y-shaped. A medial ridge 116 and two oblique-lateral ridges 117 are shown (e.g., an antero-lateral ridge and a postero-lateral ridge). Each ridge 116, 117 may have a rectangular cross-sectional profile which may have its longest dimension oriented radially outwardly relative to a longitudinal centerline 132. The medial ridge 116 may include a superimposed longitudinal groove 158. Holes 160 may extend through the ridges 116, 117 near the proximal body 110. Sutures, cables, or other lines may be routed through the holes 160 to re-attach soft tissues or bone fragments to the humerus. A lateral groove 118 and two oblique-medial grooves 119 are shown (e.g., an antero-medial groove and a postero-medial groove). The ridges 116, 117 may merge together along some or all of the length of the distal shaft 112 to form a central longitudinal solid portion 120. The central longitudinal solid portion 120 may track along, or may define, the longitudinal centerline 132. The longitudinal centerline 132 may be straight or linear, or it may be curved, bent, irregular, and so on. Referring to FIG. 1E, at least the distal portion of the longitudinal centerline 132 may be straight or linear in a posterior (or anterior) view. Referring to FIGS. 1D and 1F, the entire longitudinal centerline 132 may be straight or linear in medial and/or lateral views.

Each ridge 116, 117 may extend transversely away from the longitudinal centerline 132 a first distance near the proximal body 110 and a second distance farther from the proximal body (e.g., closer to the free end). For each ridge 116, 117 the second distance may be less than the first distance. The ridges 116, 117 and/or grooves 118, 119 may also be wider near the proximal end 10 of the distal shaft 112 and narrower near the distal end 12. The proximal and distal distances and/or the proximal and distal transverse widths for each ridge 116, 117 may be the same as, or different from, the corresponding proximal and distal distances of the other ridges of the distal shaft 112. In the example shown, with three ridges 116, 117, there may be three unique proximal distances, three unique distal distances, three unique proximal transverse widths, and/or three unique distal transverse widths. In the example shown, the antero-lateral and postero-lateral ridges 117 may be identical mirror images of each other, while the medial ridge 116 may be different from the ridges 117. The differences may be more pronounced near the proximal body 110. Optionally, the distal distances may be equal and/or the distal widths may be equal.

Referring to FIG. 1E, humeral stem 100 may include one or more regions with specialized surface structure, and/or one or more different types of surface structure. A first surface structure region 162 may be on the exterior of the proximal body 110, in other words, proximal to dashed line 164. A second surface structure region 166 may be on the proximal end of the distal shaft 112, in other words, between dashed lines 164 and 168. Preferably, the first surface structure region 162 may include a rough structure suitable to provide initial stability when the proximal body 110 is impacted into a prepared bone socket, and the second surface structure region 166 may include a surface structure conducive to bone ongrowth/ingrowth to promote long-term fixation. The location and extent of the first and second surface structure regions 162, 166 may be specified so as to achieve particular short and long term fixation objectives.

FIGS. 2A-2D illustrate various views of a ball or convex articular component 200, according to an embodiment of the present disclosure.

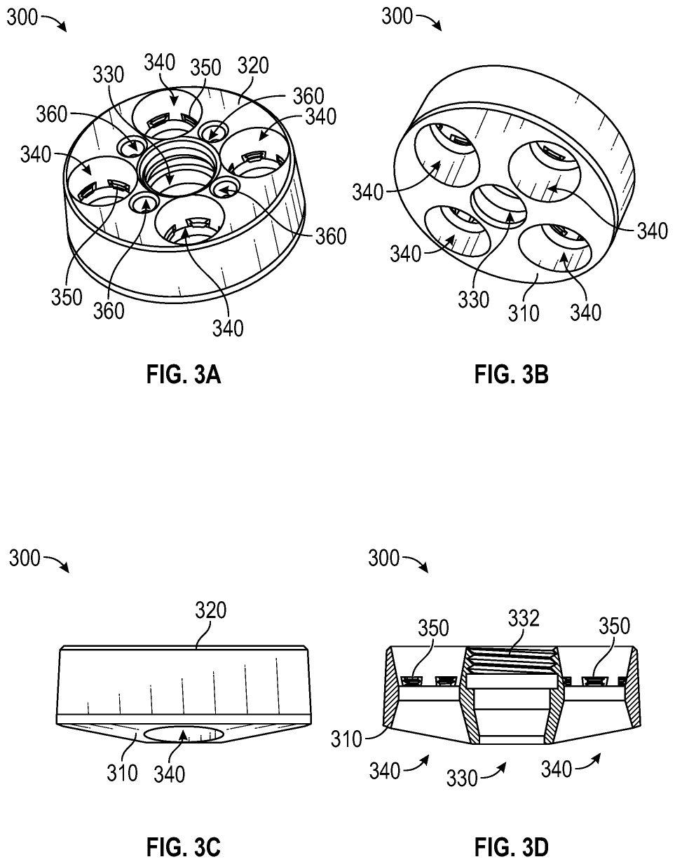

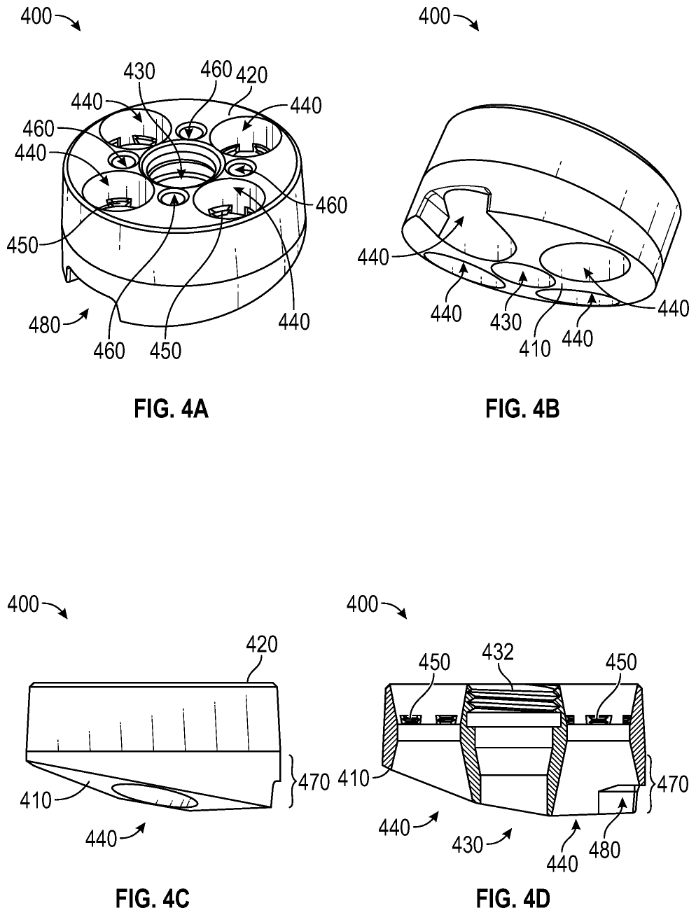

The convex articular component 200 may be utilized with the humeral stem 100 shown in FIGS. 1A-1F for an anatomical shoulder arthroplasty procedure. Conversely, the convex articular component 200 may be utilized with the baseplate 300 and/or the augmented baseplate 400 shown in FIGS. 3A-4D for a reverse shoulder arthroplasty procedure. Likewise, a concave articular component (similar in shape to that shown in FIGS. 17A-17D) may be utilized with the humeral stem 100 for a reverse shoulder arthroplasty procedure. Conversely, the concave articular component may be utilized with the baseplate 300 and/or the augmented baseplate 400 shown in for an anatomical shoulder arthroplasty procedure.

The convex articular component 200 may generally include a convex articular surface 222 and a connection interface 230.

In some embodiments, the convex articular surface 222 may comprise a hemispherical shape.

The convex articular surface 222 may be shaped to articulate with a glenoid cavity/socket or a glenoid implant (such as a concave articular component having a shape similar to that shown in FIGS. 17A-17D).

In some embodiments, the connection interface 230 may include a recess interconnection 232, a threaded portion 234, and an access passageway 236.

In some embodiments, the recess interconnection 232 may include a female taper shape that may create a taper lock with a male taper shape of the baseplate 300 and/or the augmented baseplate 400 (e.g., see FIGS. 3A-4D).

In some embodiments, the convex articular component 200 may be further secured to the baseplate 300 and/or the augmented baseplate 400 with a screw or a set screw (not shown) that may be received within the threaded portion 234.

In some embodiments, the threaded portion 234 may also be utilized to couple with an insertion tool or a removal tool (not shown).

In some embodiments, the screw or set screw may be accessed via the access passageway 236 with a driver tool (not shown) to removably couple the convex articular component 200 with the baseplate 300 and/or the augmented baseplate 400.

In other embodiments, the connection interface 230 may include a stem interconnection (not shown) with geometry shaped to removably couple the convex articular component 200 with the interior socket 122 inside the proximal body 110 of the humeral stem 100.

In some embodiments, the stem interconnection may utilize the holes 124, 126, 128 of the humeral stem 100 to receive at least a portion of the stem interconnection.

In some embodiments, the convex articular component 200 may be interchangeable with a concave articular component (such as a concave articular component having a shape similar to that shown in FIGS. 17A-17D) to select between an anatomical or reverse shoulder arthroplasty procedure.

In some embodiments, the convex articular component 200 may include a chamfered or beveled surface 240 to reduce unintended contact with surrounding soft tissues in a region of the convex articular component 200 that may be in limited contact with the convex articular surface 222.

FIGS. 3A-3D illustrate various views of a baseplate 300, according to an embodiment of the present disclosure. Specifically, FIG. 3A is an oblique view of the baseplate 300, FIG. 3B is another oblique view of the baseplate 300, FIG. 3C is a side view of the baseplate 300, and FIG. 3D is a cross-sectional view of the baseplate 300 of FIG. 3C.

The baseplate 300 may generally include a bone-facing side 310, a superior side 320 opposite the bone-facing side 310, a central fastener passageway 330, one or more peripheral fastener passageways 340, one or more retention features 350, and one or more instrument passageways 360 to couple with an instrument (not shown), such as an inserter tool, an extractor tool, etc.

In some embodiments, the bone-facing side 310 may include a surface having a convex shape, as shown in FIGS. 3C and 3D.

In some embodiments, the bone-facing side 310 may include a surface having a flat shape (not shown).

In some embodiments, the bone-facing side 310 may include a surface having a concave shape (not shown).

In some embodiments, the bone-facing side 310 may include one or more regions with specialized surface structure (e.g., a rough structure, etc.) and/or one or more different types of surface structures (not shown).

In some embodiments, the surface structure may be conducive to bone ongrowth/ingrowth to promote long-term fixation.

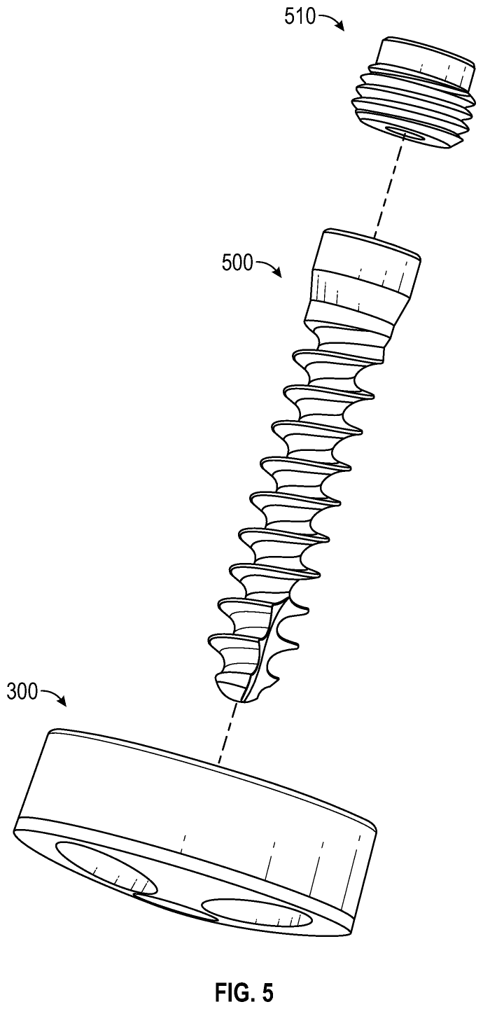

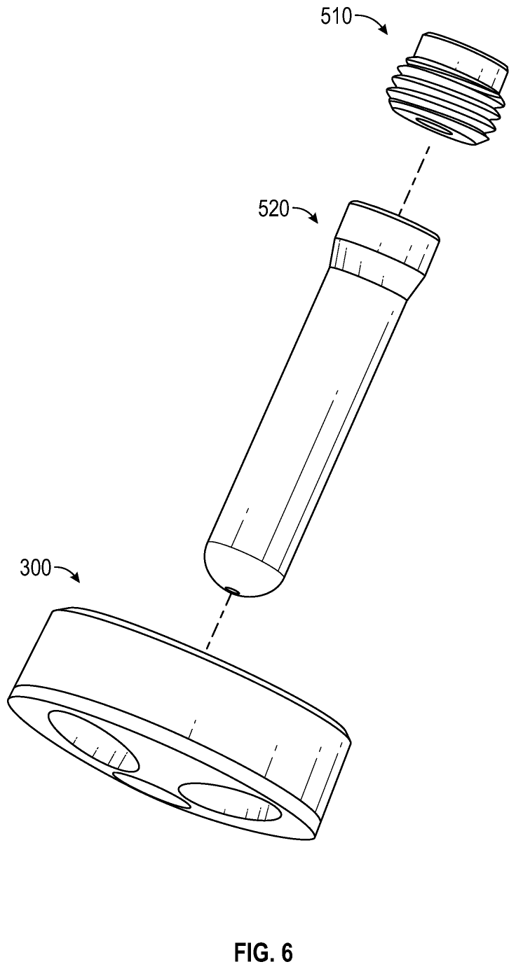

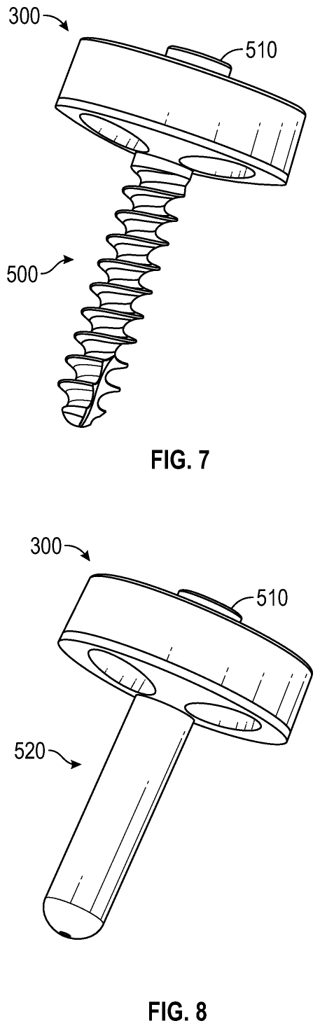

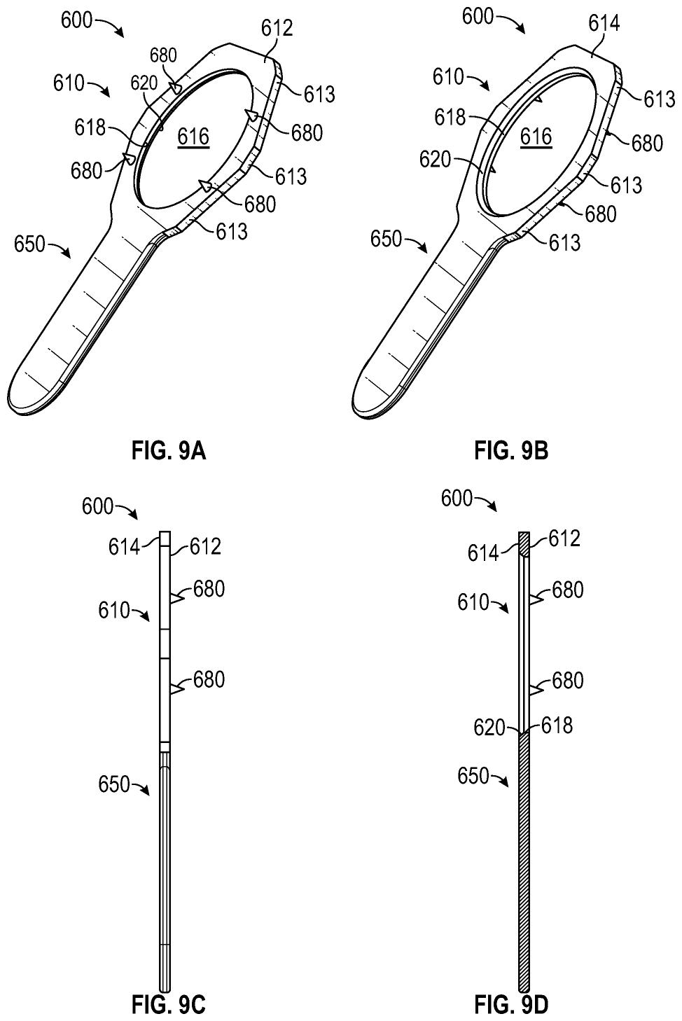

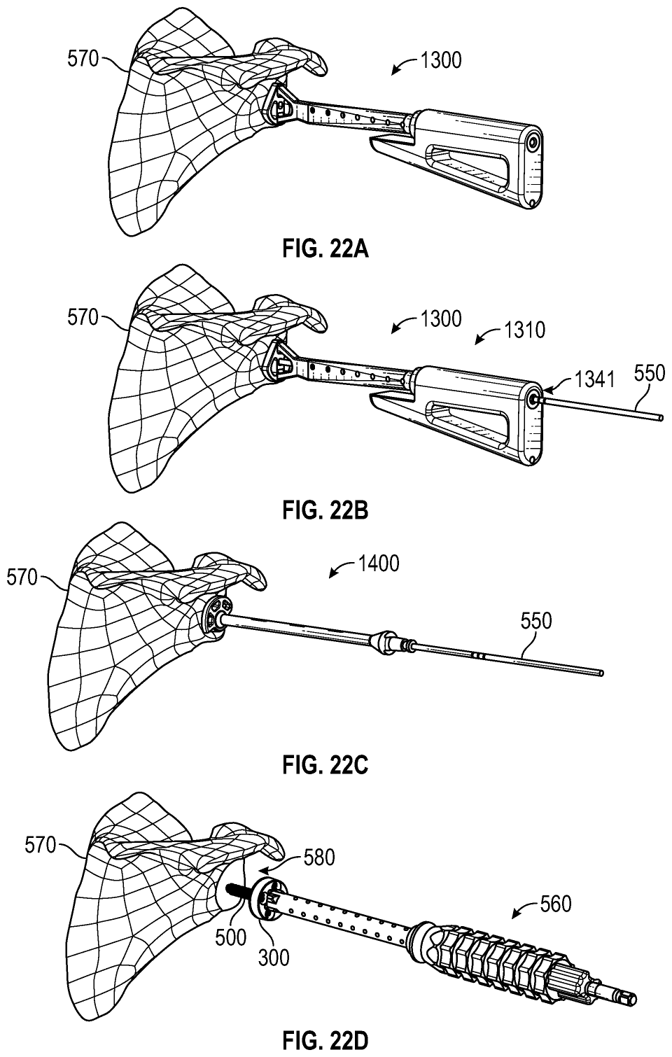

In some embodiments, the central fastener passageway 330 may be configured to receive a bone screw 500 or a post 520 therethrough (e.g., see FIGS. 5-8) to secure the baseplate 300 to a prepared glenoid cavity 580 of a scapula 570 (e.g., see FIG. 22D).

In some embodiments, the central fastener passageway 330 may include threading 332 to receive a retention member 510 therein (e.g., a set screw, a locking cap, etc.) to lock/secure the bone screw 500 or post 520 to the baseplate 300 and prevent loosening, backing-out, etc., (e.g., see FIGS. 5-8).

In some embodiments, the one or more peripheral fastener passageways 340 may be configured to receive additional fasteners or bone screws therethrough (not shown) to provide additional fixation of the baseplate 300 to a prepared glenoid cavity 580.

In some embodiments, the one or more retention features 350 may be configured to prevent loosening/backing-out of the additional fasteners from the one or more peripheral fastener passageways 340.

FIGS. 4A-4D illustrate various views of an augmented baseplate 400, according to another embodiment of the present disclosure. Specifically, FIG. 4A is an oblique view of the augmented baseplate 400, FIG. 4B is another oblique view of the augmented baseplate 400, FIG. 4C is a side view of the augmented baseplate 400, and FIG. 4D is a cross-sectional view of the augmented baseplate 400 of FIG. 4C.

The augmented baseplate 400 may generally include a bone-facing side 410, a superior side 420 opposite the bone-facing side 410, a central fastener passageway 430, one or more peripheral fastener passageways 440, one or more retention features 450, and one or more instrument passageways 460 to couple with an instrument (not shown), such as an inserter tool, an extractor tool, etc.

In some embodiments, the bone-facing side 410 may include a surface having a convex shape, as shown in FIGS. 4C and 4D.

In some embodiments, the bone-facing side 410 may include a surface having a flat shape (not shown).

In some embodiments, the bone-facing side 410 may include a surface having a concave shape (not shown).

In some embodiments, the bone-facing side 410 may include an enlarged or augmented portion 470 on at least one end of the augmented baseplate 400. The additional wedge geometry of the augmented portion 470 can provide additional material/structure to fill bony voids, bony deformations, weak bone, etc.

In some embodiments, the augmented portion 470 may include a notch 480 formed therein.

In some embodiments, the bone-facing side 410 may include one or more regions with specialized surface structure (e.g., a rough structure, etc.) and/or one or more different types of surface structures (not shown).

In some embodiments, the surface structure may be conducive to bone ongrowth/ingrowth to promote long-term fixation.

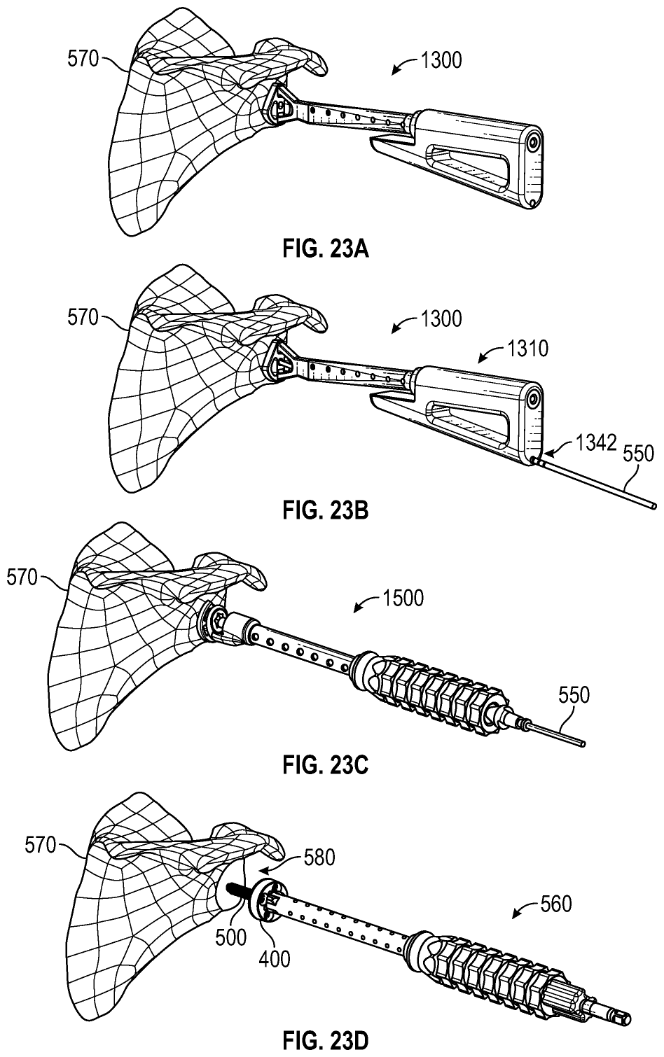

In some embodiments, the central fastener passageway 430 may be configured to receive the bone screw 500 or post 520 therethrough (e.g., see FIGS. 5-8) to secure the augmented baseplate 400 to a prepared glenoid cavity 580 of a scapula 570 (e.g., see FIG. 23D).

In some embodiments, the central fastener passageway 430 may include threading 432 to receive the retention member 510 to lock/secure the bone screw 500 or post 520 to the augmented baseplate 400 and prevent loosening, backing-out, etc., (e.g., see FIGS. 5-8).

In some embodiments, the one or more peripheral fastener passageways 440 may be configured to receive additional fasteners or bone screws therethrough (not shown) to provide additional fixation of the augmented baseplate 400 to a prepared glenoid cavity 580.

In some embodiments, the one or more retention features 450 may be configured to prevent loosening/backing-out of the additional fasteners from the one or more peripheral fastener passageways 440.

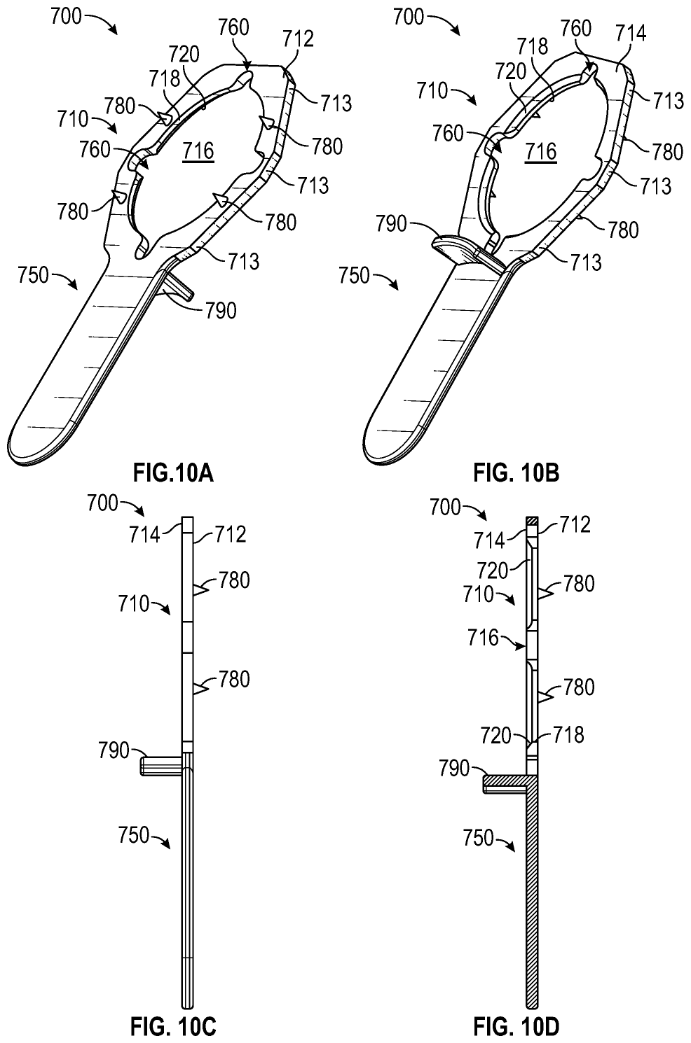

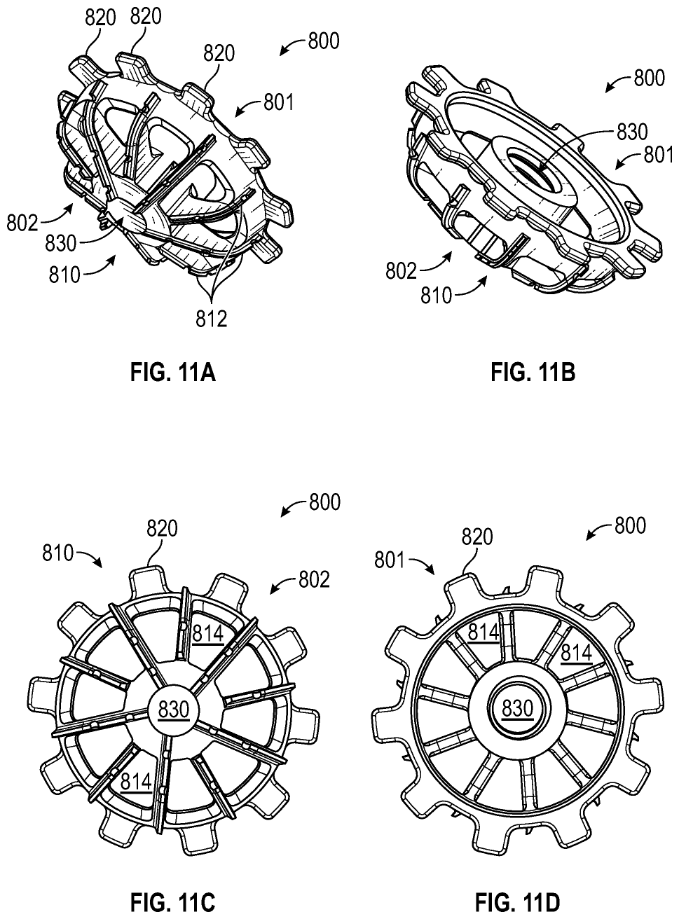

FIGS. 9A-9D illustrate various views of reamer guide plate 600, according to an embodiment of the present disclosure. Specifically, FIG. 9A is an oblique view of the reamer guide plate 600, FIG. 9B is another oblique view of the reamer guide plate 600, FIG. 9C is a side view of the reamer guide plate 600, and FIG. 9D is a cross-sectional view of the reamer guide plate 600 shown in FIG. 9C.

The reamer guide plate 600 may generally include a support ring or guide plate body 610, a handle 650, and one or more fixation elements 680.

In some embodiments, the guide plate body 610 may include a bone-facing surface 612, a superior surface 614 opposite the bone-facing surface 612, and one or more side walls 613 intermediate the bone-facing surface 612 and the superior surface 614. The guide plate body 610 may also include a reamer guide bore 616 formed through the guide plate body 610 and oriented to pass through the superior surface 614 and the bone-facing surface 612 of the guide plate body 610.

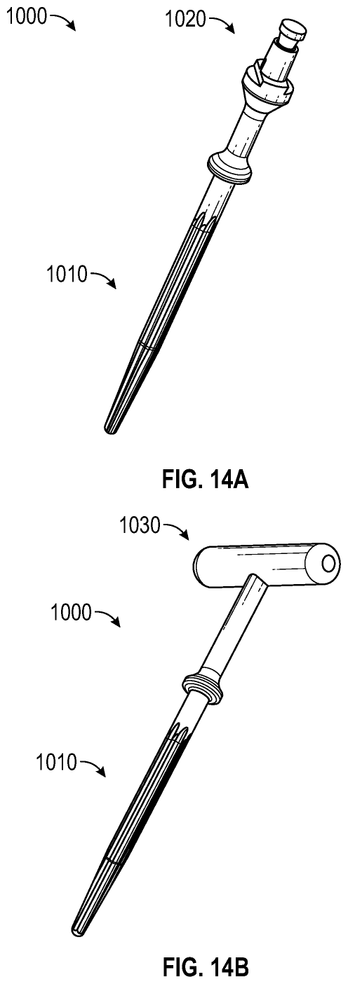

In some embodiments, the reamer guide bore 616 may be shaped to receive and guide a reamer head 800 that is placed through the reamer guide bore 616 (e.g., see FIGS. 12B and 12C).

In some embodiments, the reamer guide bore 616 may comprise a circular shape.

In some embodiments, the reamer guide bore 616 may at least be partially defined by an annular inner side wall 618 intermediate the bone-facing surface 612 and the superior surface 614.

In some embodiments, the reamer guide bore 616 may also include one or more beveled surfaces 620 intermediate the annular inner side wall 618 and the superior surface 614 of the guide plate body 610.

In some embodiments, the one or more beveled surfaces 620 of the reamer guide bore 616 may be shaped to contact one or more beveled surfaces (not shown) about a proximal end of a reamer head to limit a predetermined depth of a distal cutting surface of the reamer head that may project from the bone-facing surface 612 of the guide plate body 610.

In some embodiments, the handle 650 may be coupled to the guide plate body 610.

In some embodiments, the handle 650 may be removably couplable with the guide plate body.

In some embodiments, the handle 650 may be integrally formed with the guide plate body.

In some embodiments, the one or more fixation elements 680 may project from the bone-facing surface 612.

In some embodiments, the one or more fixation elements 680 may be configured to couple the bone-facing surface 612 of the reamer guide plate 600 to a bone, and/or help maintain stability of the reamer guide plate 600 relative to the bone during the reaming process.

In some embodiments, the one or more fixation elements 680 may comprise one or more spikes, barbs, pegs, etc., that project from the bone-facing surface 612 of the guide plate body 610.

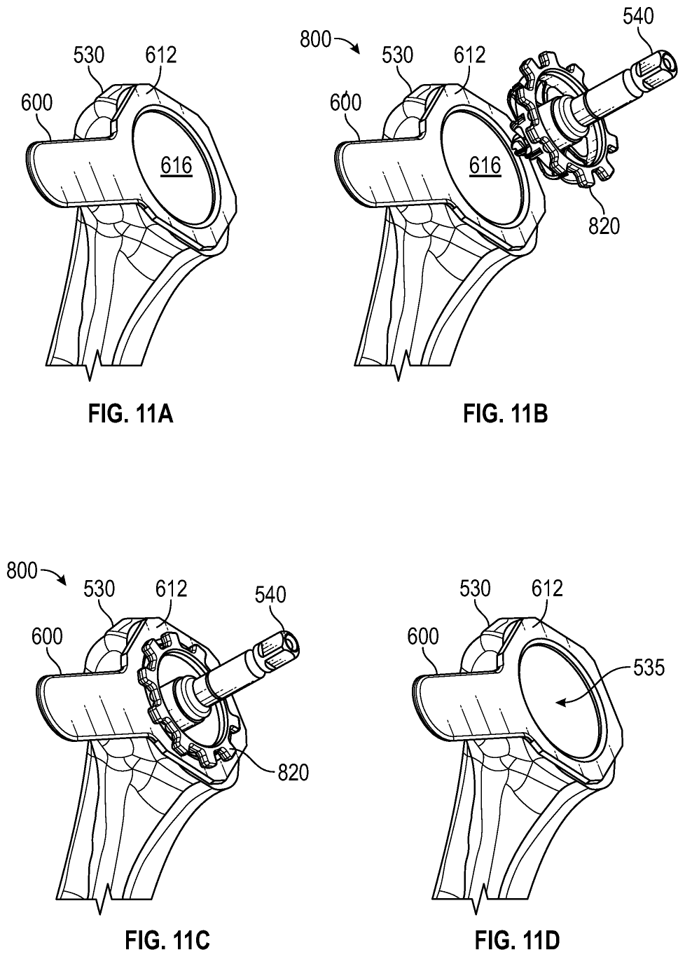

FIGS. 10A-10D illustrate various views of reamer guide plate 700, according to another embodiment of the present disclosure. Specifically, FIG. 10A is an oblique view of the reamer guide plate 700, FIG. 10B is another oblique view of the reamer guide plate 700, FIG. 10C is a side view of the reamer guide plate 700, and FIG. 10D is a cross-sectional view of the reamer guide plate 700 shown in FIG. 10C.

The reamer guide plate 700 may generally include a support ring or guide plate body 710, a handle 750, and one or more fixation elements 780.

In some embodiments, the guide plate body 710 may include a bone-facing surface 712, a superior surface 714 opposite the bone-facing surface 712, and one or more side walls 713 intermediate the bone-facing surface 712 and the superior surface 714. The guide plate body 710 may also include a reamer guide bore 716 formed through the guide plate body 710 and oriented to pass through the superior surface 714 and the bone-facing surface 712 of the guide plate body 710.

In some embodiments, the reamer guide bore 716 may be shaped to receive and guide a reamer head 800 that is placed through the reamer guide bore 716 (e.g., see FIGS. 12B and 12C).

In some embodiments, the reamer guide bore 716 may comprise an at least partially a circular shape.

In some embodiments, the reamer guide bore 716 may include one or more windows 760 formed through the guide plate body 710 about the reamer guide bore 716.

In some embodiments, the one or more windows 760 may comprise one or more lobe shapes adjacent the reamer guide bore 716, as shown in FIGS. 10A and 10B. However, it will be understood that the one or more windows 760 may comprise any shape, and the one or more windows 760 may be placed adjacent the reamer guide bore 716 or spaced apart from the reamer guide bore 716 according to any desired pattern. In this manner, the one or more windows 760 may be configured to increase visualization of a bone to help determine an optimal position for the reamer guide plate 700 relative to the bone.

In some embodiments, the reamer guide bore 716 may at least be partially defined by an annular inner side wall 718 intermediate the bone-facing surface 712 and the superior surface 714.

In some embodiments, the reamer guide bore 716 may also include one or more beveled surfaces 720 intermediate the annular inner side wall 718 and the superior surface 714 of the guide plate body 710.

In some embodiments, the one or more beveled surfaces 720 of the reamer guide bore 716 may be shaped to contact one or more beveled surfaces (not shown) disposed about a proximal end of a reamer head in order to limit a predetermined depth of a distal cutting surface of the reamer head projecting from the bone-facing surface 712 of the guide plate body 710.

In some embodiments, the handle 750 may be coupled to the guide plate body 710.

In some embodiments, the handle 750 may be removably couplable with the guide plate body.

In some embodiments, the handle 750 may be integrally formed with the guide plate body.

In some embodiments, the one or more fixation elements 780 may project from the bone-facing surface 712.

In some embodiments, the one or more fixation elements 780 may be configured to couple the bone-facing surface 712 of the reamer guide plate 700 to a bone, and/or help maintain stability of the reamer guide plate 700 relative to the bone during the reaming process.

In some embodiments, the one or more fixation elements 780 may comprise one or more spikes, barbs, pegs, etc., that project from the bone-facing surface 712 of the guide plate body 710.

In some embodiments, the reamer guide plate 700 may include at least one guard member 790 coupled with the reamer guide plate 700 and/or the handle 750. In this manner, the at least one guard member 790 may serve as a safety mechanism to keep the user's fingers/hands away from the reaming area.

In some embodiments, the at least one guard member 790 may project from the reamer guide plate 700 and/or the handle 750 superiorly.

In some embodiments, the at least one guard member 790 may project from the reamer guide plate 700 and/or the handle 750 inferiorly.

In some embodiments, the at least one guard member 790 may project from the reamer guide plate 700 and/or the handle laterally and/or medially.

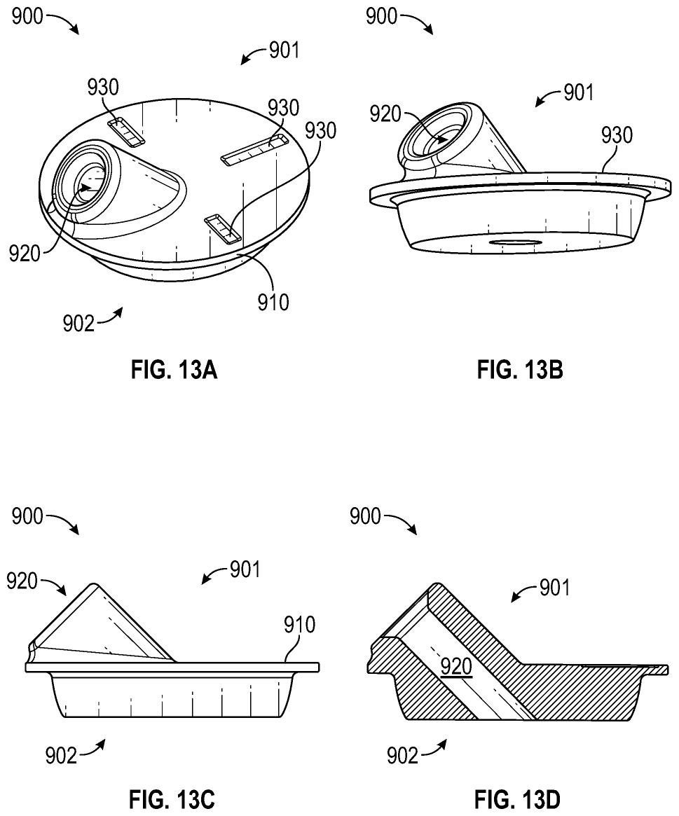

FIGS. 11A-11D illustrate various views of reamer head 800, according to an embodiment of the present disclosure. Specifically, FIG. 11A is an oblique view of the reamer head 800, FIG. 11B is another oblique view of the reamer head 800, FIG. 11C is a distal end view of the reamer head 800, and FIG. 11D is a proximal end view of the reamer head 800.

The reamer head 800 may generally include a proximal end 801, a distal end 802, a distal cutting surface 810 comprising one or more cutting features 812 and/or one or more openings 814 intermediate the one or more cutting features 812, one or more stop members 820 coupled to the proximal end 801 of the reamer head 800, and a torque connection interface 830.

In some embodiments, the one or more stop members 820 may be configured to contact the superior surface 614, 714 of the guide plate body 610, 710 to limit a predetermined depth of the distal cutting surface 810 that may project from the bone-facing surface 612, 712 of the guide plate body 610, 710 (e.g., see FIG. 12C).

In some embodiments, the one or more stop members 820 may include a plurality of tabs projecting from the proximal end 801 of the reamer head 800, as shown in FIGS. 11A-11D.

In some embodiments, the one or more stop members 820 may include a first beveled surface (not shown) circumscribing the proximal end 801 of the reamer head 800, and the reamer guide plate 600, 700 may include at least one second beveled surface shaped to contact the first beveled surface of the reamer head 800 to limit a predetermined depth of the distal cutting surface 810 projecting from the bone-facing surface 612, 712 of the guide plate body 610, 710 (e.g., see FIG. 12C).

In some embodiments, the one or more openings 814 intermediate the one or more cutting features 812 may be configured to capture/gather bone chips during the reaming process.

In some embodiments, the torque connection interface 830 may be configured to removably couple with a reamer head driver 540 (e.g., see FIGS. 12B and 12C).

FIGS. 12A-12D illustrate various views of an example reaming procedure for a humerus 530 or proximal humerus utilizing the reamer guide plate 600, the reamer head 800, and the reamer head driver 540, according to an embodiment of the present disclosure. Specifically, FIG. 12A shows the reamer guide plate 600 coupled to the humerus 530; FIG. 12B shows the reamer head 800 above the humerus 530 prior to the reaming procedure; FIG. 12C shows the reamer head 800 inserted into the humerus 530 to perform the reaming procedure; and FIG. 12D shows the humerus 530 after the reaming procedure has been performed to form a bone cavity 535.

In some embodiments, a method/procedure for reaming a bone may include, placing a bone-facing surface 612, 712 of a reamer guide plate 600, 700 against the bone, inserting a distal cutting surface 810 of a reamer head 800 through a reamer guide bore 616, 716 formed through the reamer guide plate 600, 700 (the reamer guide bore 616, 716 shaped to receive and guide the distal cutting surface 810 placed therethrough), and reaming the bone with the distal cutting surface 810 of the reamer head 800 to form a bone cavity 535 in the bone having a predetermined depth relative to the bone-facing surface 612, 712 of the reamer guide plate 600, 700. In this manner, the bone cavity 535 may have the appropriate depth, and may also be concentric with the reamer guide bore 616, 716. Thus, without the reamer guide plate 600, 700, the reamer head 800 may tend to wander or move within the bone during the reaming process. This motion can have an adverse effect on the fixation of the humeral stem 100 within the prepared bony surfaces (for example, when press-fit or interference fit fixation is desired between the humeral stem 100 and the humerus 530).

In some embodiments, the method/procedure may also include coupling the bone-facing surface 612, 712 of the reamer guide plate 600, 700 to the bone by coupling one or more fixation elements 680, 780 of the reamer guide plate 600, 700 to the bone, as previously discussed.

In some embodiments of the method/procedure, coupling the bone-facing surface 612, 712 of the reamer guide plate 600, 700 to the bone may include pressing one or more spikes, barbs, pegs, etc., that project from the bone-facing surface 612, 712 into the bone.

In some embodiments, the method/procedure may also include limiting the predetermined depth of the bone cavity 535 relative to the bone-facing surface 612, 712 of the reamer guide plate 600, 700 by contacting one or more stop members 820 coupled to a proximal end 801 of the reamer head 800 with a superior surface 614, 714 of the reamer guide plate 600, 700 in order to limit the predetermined depth of the distal cutting surface 810 that projects from the bone-facing surface 612, 712 of the reamer guide plate 600, 700.

In some embodiments of the method/procedure, the one or more stop members 820 may include a plurality of tabs projecting from the proximal end 801 of the reamer head 800.

In some embodiments of the method/procedure, the one or more stop members 820 may include a first beveled surface (not shown) circumscribing the proximal end 801 of the reamer head 800. The reamer guide plate 600, 700 may also include at least one second beveled surface shaped to contact the first beveled surface of the reamer head 800 in order to limit the predetermined depth of the distal cutting surface 810 that projects from the bone-facing surface 612, 712.

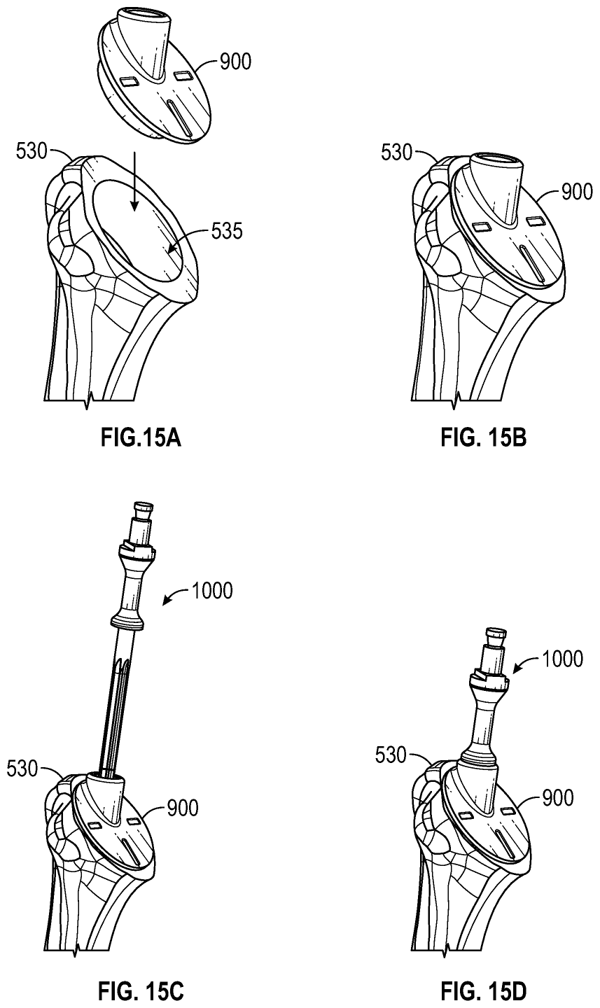

FIGS. 13A-13D illustrate various views of a humeral drill guide 900, according to an embodiment of the present disclosure. Specifically, FIG. 13A is an oblique view of the humeral drill guide 900, FIG. 13B is another oblique view of the humeral drill guide 900, FIG. 13C is a side view of the humeral drill guide 900, and FIG. 13D is a cross-sectional view of the humeral drill guide 900 of FIG. 13C.

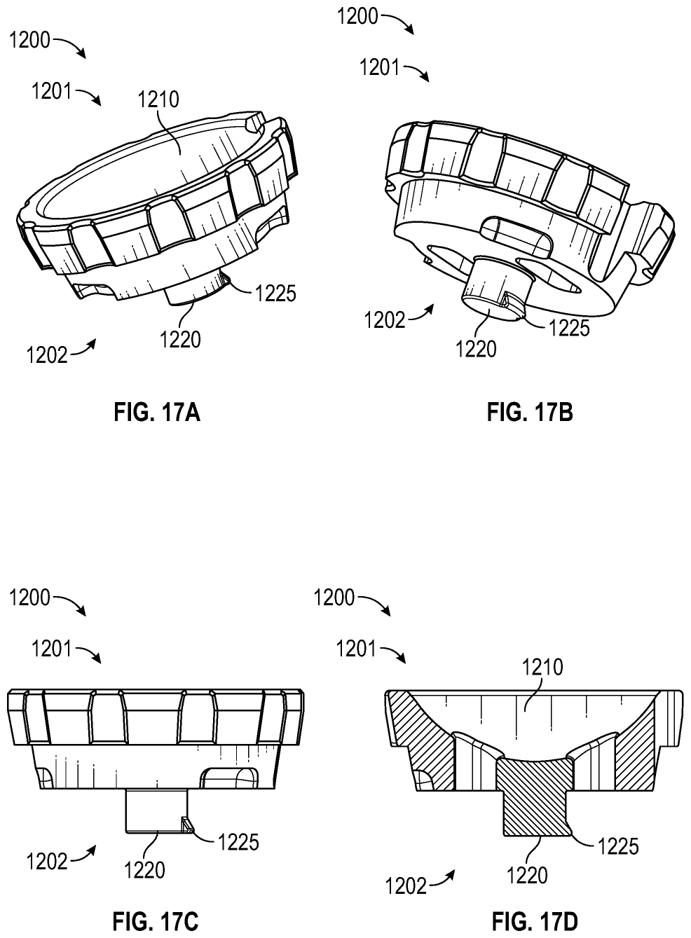

The humeral drill guide 900 may generally include a proximal end 901, a distal end 902, an alignment flange 910, a humeral drill guide bore 920, and one or more orientation makings 930. Operation of the humeral drill guide 900 will be discussed below with reference to FIGS. 15A-15D.

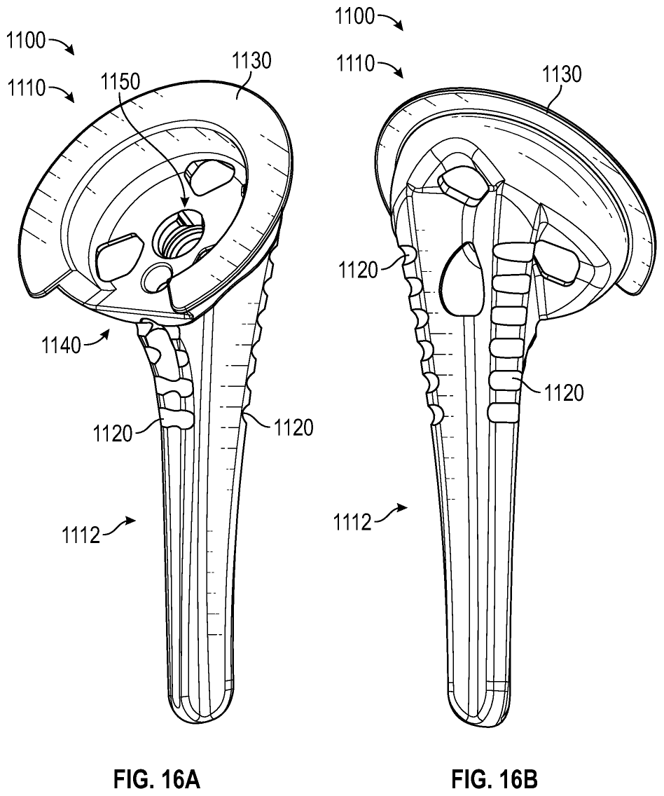

FIG. 14A illustrates an oblique view of a humeral punch tool or humeral drill tool 1000 including a drill portion 1010 and a torque connection interface 1020 (e.g., for powered operation). FIG. 14B illustrates an oblique view of the humeral drill tool 1000 of FIG. 14A with a T-handle 1030 in place of the torque connection interface 1020 (e.g., for manual operation).

FIGS. 15A-15D illustrate various views of an example drilling procedure for a humerus 530 or proximal humerus to prepare the humerus 530 to receive the distal shaft 112 of the humeral stem 100 shown in FIGS. 1A-1F. The drilling procedure may utilize the humeral drill guide 900 and the humeral drill tool 1000 shown in FIGS. 13A-14B. Specifically, FIG. 15A is an oblique view of the humeral drill guide 900 being inserted into the bone cavity 535; FIG. 15B is an oblique view of the humeral drill guide 900 aligned with, and seated within, the bone cavity 535; FIG. 15C is an oblique view of the humeral drill tool 1000 of FIG. 14A being inserted through the humeral drill guide bore 920 and into an intramedullary canal of the humerus 530; and FIG. 15D is an oblique view of the humeral drill tool 1000 fully inserted into the intramedullary canal of the humerus 530 to perform the drilling procedure.

In some embodiments, the distal end 902 of the humeral drill guide 900 may have a bone-facing geometry that closely matches that of the bone cavity 535 to help correctly align the humeral drill guide 900 within the bone cavity 535.

In some embodiments, the alignment flange 910 of the humeral drill guide 900 may also act as a depth stop against the humerus 530 to help correctly align the humeral drill guide bore 920 relative to an intramedullary canal of the humerus 530.

In some embodiments, the one or more orientation makings 930 may also help a user place the humeral drill guide 900 in the proper orientation relative to the humerus 530.

Once the humeral drill guide 900 has been placed within the bone cavity 535 and properly aligned relative to the humerus 530, the humeral drill tool 1000 may be inserted through the humeral drill guide bore 920 and into the intramedullary canal of the humerus 530 to perform the drilling operation, as shown in FIGS. 15C and 15D. In this manner, the location of the humeral drill guide bore 920 may match the trajectory of the distal shaft 112 of the humeral stem 100 to be received within the prepared intramedullary canal of the humerus 530.

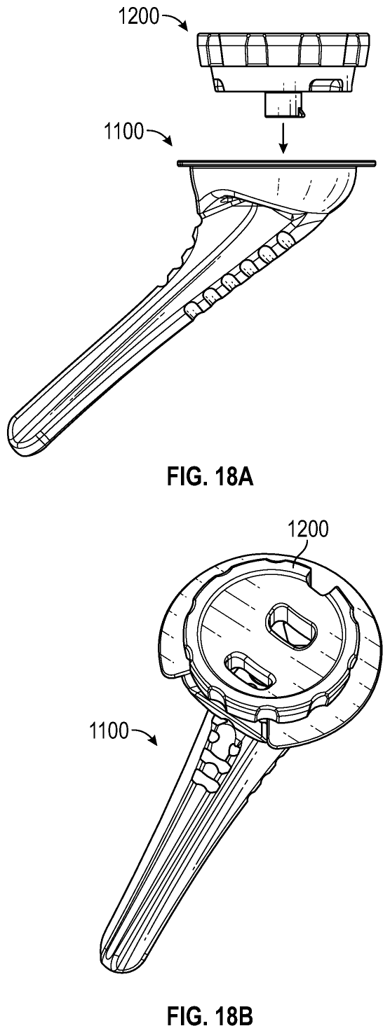

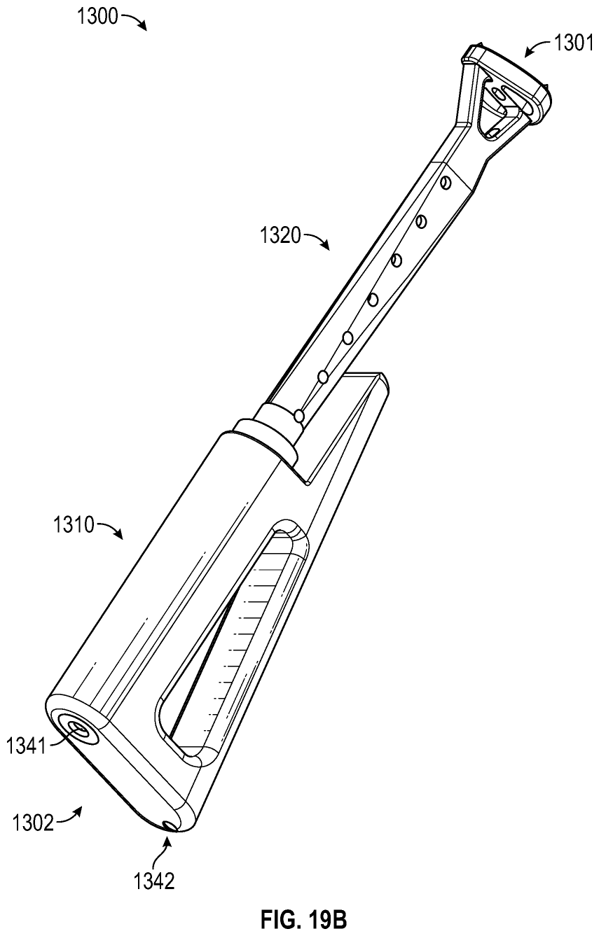

FIGS. 16A and 16B show oblique views of a broach or trial humeral stem 1100, according to an embodiment of the present disclosure. The trial humeral stem 1100 may generally include a proximal body 1110 and a distal shaft 1112. The distal shaft 1112 may have the same basic shape as the distal shaft 112 of the humeral stem 100, previously discussed with reference to FIGS. 1A-1F. However, the trial humeral stem 1100 may be slightly undersized in comparison to the humeral stem 100 and may also include additional features, such as one or more broaching serrations 1120 formed in the ridges/columns, a proximal depth stop or proximal flange 1130 disposed about the proximal body 1110, a cut out portion 1140 on the medial side of the proximal body 1110, a tab slot 1150, and an internal cannulation (not shown) which may be configured to couple with an insertion/removal instrument (not shown) and/or with trial bearing surface implants, as shown in FIGS. 18A and 18B.

In some embodiments, the proximal flange 1130 may ensure the trial humeral stem 1100 is inserted into the intramedullary canal of the humerus 530 to an appropriate depth.

In some embodiments, the cut out portion 1140 may reduce the profile of the proximal body 1110 and minimize disruption to the precisely prepared humeral bone during the trialing process.

In some embodiments, the tab slot 1150 may receive an extension tab of a trial bearing surface implant (e.g., see the extension tab 1225 of the trial concave articular component 1200 shown in FIGS. 17A-17D) in order to form a “twist-to-lock” type locking mechanism between the trial humeral stem 1100 and the trial bearing surface implant as the extension tab rotates within the tab slot 1150 and is captured against the trial humeral stem 1100, as shown in FIGS. 18A and 18B.

FIGS. 17A-17D illustrate various views of a trial concave articular component 1200, according to an embodiment of the present disclosure. Specifically, FIG. 17A is an oblique view of the trial concave articular component 1200, FIG. 17B is another oblique view of the trial concave articular component 1200, FIG. 17C is a side view of the trial concave articular component 1200, and FIG. 17D is a cross-sectional view of the trial concave articular component 1200 of FIG. 17C.

The trial concave articular component 1200 may generally include a proximal end 1201 comprising a concave articular surface 1210, and a distal end 1202 comprising a post 1220 having an extension tab 1225 that may protrude therefrom.

The trial concave articular component 1200 may be secured to the trial humeral stem 1100 via the extension tab 1225 of the trial concave articular component 1200 rotating within the tab slot 1150 of the trial humeral stem 1100 to form the “twist-to-lock” type locking mechanism previously discussed and shown in FIGS. 18A and 18B. In this manner, different sizes of trial concave articular components may be attached to the trial humeral stem 1100 to find which size may be best for a particular arthroplasty procedure.

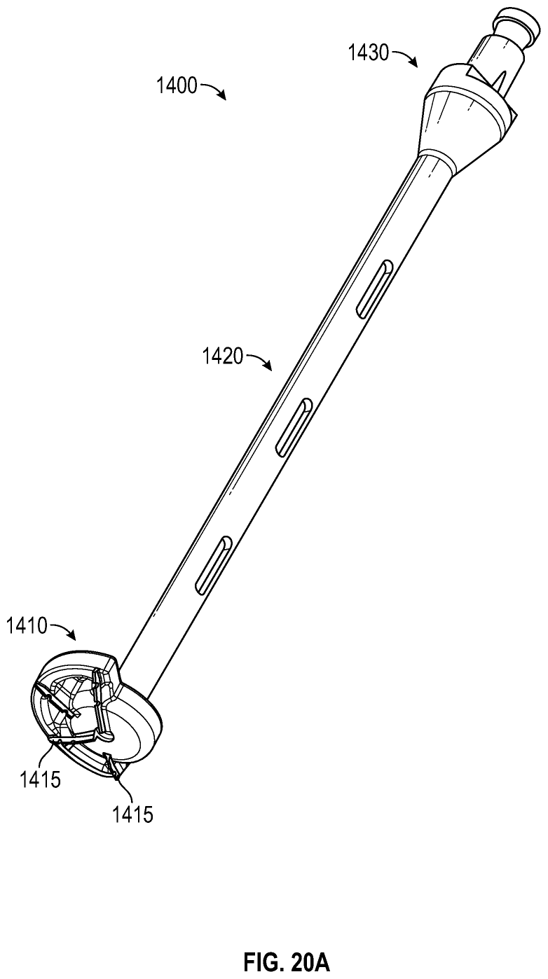

FIGS. 19A-23D illustrate various instruments and procedure steps for preparing a scapula to receive the baseplate 300 of FIGS. 3A-3D and/or the augmented baseplate of FIGS. 4A-4D.

FIGS. 19A and 19B illustrate oblique views of a glenoid drill guide 1300, according to an embodiment of the present disclosure. The glenoid drill guide 1300 may generally include a proximal end 1301 comprising a handle 1310 with at least one pin or drill bit passageway formed therethrough, an intermediate shaft portion 1320, and a distal end 1302 comprising a glenoid-facing surface 1350 with one or more fixation members 1330 projecting therefrom and a central hole 1360 formed therethrough.

In some embodiments, the handle 1310 may include a first drill bit passageway 1341 formed therethrough and configured to guide a drill bit 550 along a first trajectory through the handle 1310, the intermediate shaft portion 1320, and out of the central hole 1360 formed in the glenoid-facing surface 1350.

In some embodiments, the handle 1310 may include a second drill bit passageway 1342 formed therethrough and configured to guide a drill bit 550 along a second trajectory through the handle 1310, through a distal part of the intermediate shaft portion 1320, and out of the central hole 1360 formed in the glenoid-facing surface 1350.

In some embodiments, the handle 1310 may include more than two drill bit passageways formed therethrough (not shown) and configured to guide a drill bit 550 along more than two trajectories and out the central hole 1360 formed in the glenoid-facing surface 1350.

In some embodiments, the first trajectory of the first drill bit passageway 1341 may be oriented at about zero degrees with respect to the central hole 1360 and/or the glenoid-facing surface 1350.

In some embodiments, the second trajectory of the second drill bit passageway 1342 may be oriented at about ten degrees with respect to the central hole 1360 and/or the glenoid-facing surface 1350.

However, it will be understood that any drill bit passageway may be formed through the handle 1310 that may include any trajectory between −90 degrees and +90 degrees with respect to the central hole 1360 and/or the glenoid-facing surface 1350.

In some embodiments, the one or more fixation members 1330 may comprise one or more spikes, barbs, pegs, etc., that project from the glenoid-facing surface 1350 of the glenoid drill guide 1300.

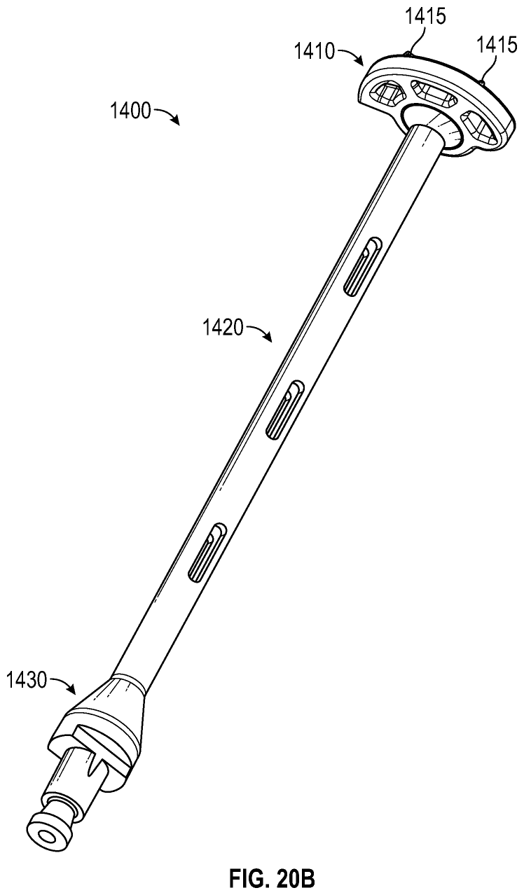

FIGS. 20A and 20B illustrate oblique views of a reamer tool 1400, according to an embodiment of the present disclosure. The reamer tool 1400 may generally include a reamer head 1410 comprising one or more cutting features 1415, a reamer shaft 1420, and a torque connection interface 1430. In some embodiments, the reamer tool 1400 may be cannulated.

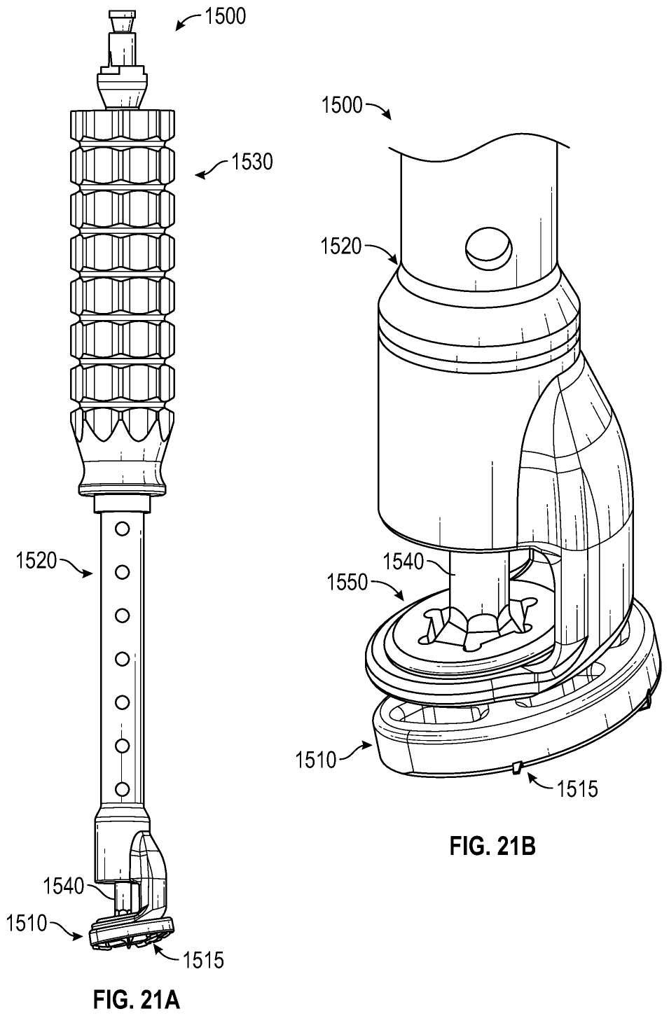

FIGS. 21A and 21B illustrates oblique views of an offset reamer tool 1500, according to another embodiment of the present disclosure. The offset reamer tool 1500 may generally include an offset reamer head 1510 comprising one or more cutting features 1515, a sleeve 1520, a handle 1530, a reamer shaft 1540, and a polyaxial torque connection interface 1550 between the reamer shaft 1540 and the offset reamer head 1510.

In some embodiments, an angle of the offset reamer head 1510 with respect to the reamer shaft may be fixed.

In some embodiments, an angle of the offset reamer head 1510 with respect to the reamer shaft may be adjustable.

In some embodiments, an angle of the offset reamer head 1510 with respect to the reamer shaft may be fixed at any angle between −90 degrees and +90 degrees.

In some embodiments, an angle of the offset reamer head 1510 with respect to the reamer shaft may be adjustable to any angle between −90 degrees and +90 degrees.

In some embodiments, the offset reamer tool 1500 may be cannulated.

FIGS. 22A-22D illustrate various views of a procedure for preparing a glenoid cavity of a scapula 570 utilizing the glenoid drill guide 1300 and reamer tool 1400 to implant the baseplate 300 of FIGS. 3A-3D on the prepared glenoid cavity 580.

In a first step of the procedure, the glenoid-facing surface 1350 of the glenoid drill guide 1300 may be placed against the glenoid cavity of the scapula 570, as shown in FIG. 22A. In some embodiments, one or more fixation members 1330 projecting from the glenoid-facing surface 1350 may be pressed into the glenoid cavity to couple/stabilize the glenoid drill guide 1300 with respect to the glenoid cavity.

In a second step of the procedure, a drill bit 550 may be inserted through a first drill bit passageway 1341 formed through the handle 1310 of the glenoid drill guide 1300 along a first trajectory and into the glenoid cavity, as shown in FIG. 22B. In some embodiments, the first trajectory may be oriented at about zero degrees with respect to a central hole 1360 and/or a glenoid-facing surface 1350 of the glenoid drill guide 1300.

In a third step of the procedure (see FIG. 22C), the glenoid drill guide 1300 may be removed from the drill bit 550, the reamer tool 1400 may be placed over the drill bit 550, and the reamer tool 1400 may be utilized to ream the glenoid cavity and form the prepared glenoid cavity 580 shown in FIG. 22D to receive the baseplate 300 of FIGS. 3A-3D.

In a fourth step of the procedure, the reamer tool 1400 may be removed and a driver tool 560 may be utilized to rotate the bone screw 500 to implant the baseplate 300 onto the prepared glenoid cavity 580, as shown in FIG. 22D.

FIGS. 23A-23D illustrate various views of a procedure for preparing a glenoid cavity of a scapula 570 utilizing the glenoid drill guide 1300 and the offset reamer tool 1500 to implant the augmented baseplate 400 of FIGS. 4A-4D on the prepared glenoid cavity 580.

In a first step of the procedure, the glenoid-facing surface 1350 of the glenoid drill guide 1300 may be placed against the glenoid cavity of the scapula 570, as shown in FIG. 23A. In some embodiments, one or more fixation members 1330 projecting from the glenoid-facing surface 1350 may be pressed into the glenoid cavity to couple/stabilize the glenoid drill guide 1300 with respect to the glenoid cavity.

In a second step of the procedure, a drill bit 550 may be inserted through a second drill bit passageway 1342 formed through the handle 1310 of the glenoid drill guide 1300 along a second trajectory and into the glenoid cavity, as shown in FIG. 23B. In some embodiments, the second trajectory may be oriented at about ten degrees with respect to a central hole 1360 and/or a glenoid-facing surface 1350 of the glenoid drill guide 1300.

In a third step of the procedure (see FIG. 23C), the glenoid drill guide 1300 may be removed from the drill bit 550, the offset reamer tool 1500 may be placed over the drill bit 550, and the offset reamer tool 1500 may be utilized to ream the glenoid cavity and form the prepared glenoid cavity 580 shown in FIG. 23D to receive the augmented baseplate 400 of FIGS. 4A-4D.

In a fourth step of the procedure, the offset reamer tool 1500 may be removed and a driver tool 560 may be utilized to rotate the bone screw 500 to implant the baseplate 300 onto the prepared glenoid cavity 580, as shown in FIG. 23D.

Any procedures/methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112(f). It will be apparent to those having skill in the art that changes may be made to the details of the above-described embodiments without departing from the underlying principles set forth herein.

The phrases “connected to,” “coupled to,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the devices, systems, instruments, and methods disclosed herein.

What is claimed is:

1. A reamer guide plate comprising:
a guide plate body comprising:
a bone-facing surface;
a superior surface, opposite the bone-facing surface; and
a reamer guide bore formed through the guide plate body and oriented to pass through the superior surface and the bone-facing surface of the guide plate body, wherein the reamer guide bore is shaped to receive and guide a reamer head that is placed therethrough;
a handle coupled to the guide plate body; and
one or more fixation elements projecting from the bone-facing surface, the one or more fixation elements configured to couple the bone-facing surface of the reamer guide plate to a bone.

2. The reamer guide plate of claim 1, wherein the reamer guide bore comprises a circular shape defined by an annular inner side wall intermediate the bone-facing surface and the superior surface.

3. The reamer guide plate of claim 2, further comprising one or more beveled surfaces intermediate the annular inner side wall and the superior surface.

4. The reamer guide plate of claim 2, further comprising one or more windows formed through the guide plate body about the reamer guide bore.

5. The reamer guide plate of claim 4, wherein the one or more windows comprise one or more lobe shapes adjacent the reamer guide bore.

6. The reamer guide plate of claim 1, wherein the one or more fixation elements comprise one or more spikes projecting from the bone-facing surface.

7. The reamer guide plate of claim 1, wherein the handle is removably couplable with the guide plate body.

8. The reamer guide plate of claim 1, wherein the handle is integrally formed with the guide plate body.

9. The reamer guide plate of claim 1, further comprising at least one guard member coupled with the reamer guide plate.

10. A bone reaming system comprising:
a reamer guide plate comprising:
a guide plate body comprising:
a bone-facing surface;
a superior surface, opposite the bone-facing surface; and
a reamer guide bore formed through the guide plate body and oriented to pass through the superior surface and the bone-facing surface of the guide plate body; and
a reamer head comprising:
a distal cutting surface comprising one or more cutting features; and
one or more stop members coupled to a proximal end of the reamer head,
wherein:
the reamer guide bore is shaped to receive and guide the distal cutting surface of the reamer head placed through the reamer guide bore; and
the one or more stop members are configured to contact the superior surface of the guide plate body to limit a predetermined depth of the distal cutting surface projecting from the bone-facing surface of the guide plate body.

11. The bone reaming system of claim 10, wherein the one or more stop members comprise a plurality of tabs projecting from the proximal end of the reamer head.

12. The bone reaming system of claim 10, wherein:
the one or more stop members comprise a first beveled surface circumscribing the proximal end of the reamer head; and
the reamer guide plate further comprises at least one second beveled surface shaped to contact the first beveled surface of the reamer head and limit the predetermined depth of the distal cutting surface projecting from the bone-facing surface of the guide plate body.

13. The bone reaming system of claim 10, wherein the reamer guide bore formed through the guide plate body comprises a circular shape defined by an annular inner side wall intermediate the bone-facing surface and the superior surface.

14. The bone reaming system of claim 10, wherein the reamer guide plate further comprises one or more windows formed through the guide plate body about the reamer guide bore.

15. A method of reaming a bone comprising:
placing a bone-facing surface of a reamer guide plate against the bone;
inserting a distal cutting surface of a reamer head through a reamer guide bore formed through the reamer guide plate, the reamer guide bore shaped to receive and guide the distal cutting surface placed therethrough; and
reaming the bone with the distal cutting surface of the reamer head to form a bone cavity having a predetermined depth relative to the bone-facing surface of the reamer guide plate.

16. The method of claim 15, further comprising:
coupling the bone-facing surface of the reamer guide plate to the bone by coupling one or more fixation elements of the reamer guide plate to the bone.

17. The method of claim 16, wherein coupling the bone-facing surface of the reamer guide plate to the bone comprises:
pressing one or more spikes that project from the bone-facing surface into the bone.

18. The method of claim 15, further comprising:
limiting the predetermined depth of the bone cavity relative to the bone-facing surface of the reamer guide plate by contacting one or more stop members coupled to a proximal end of the reamer head with a superior surface of the reamer guide plate to limit the predetermined depth of the distal cutting surface projecting from the bone-facing surface of the reamer guide plate.

19. The method of claim 18, wherein the one or more stop members comprise a plurality of tabs projecting from the proximal end of the reamer head.

20. The method of claim 18, wherein:
the one or more stop members comprise a first beveled surface circumscribing the proximal end of the reamer head; and
the reamer guide plate further comprises at least one second beveled surface shaped to contact the first beveled surface of the reamer head and limit the predetermined depth of the distal cutting surface projecting from the bone-facing surface of the reamer guide plate.
